(19) United States
(12) Patent Application Publication
(10) Pub. No.: US 2021/0308147 A1
(43) Pub. Date: Oct. 7, 2021

(54) USE OF ANDROGENS FOR TREATING CORONA VIRUS

(71) Applicant: Edward M. Lichten, Birmingham, MI (US)

(72) Inventor: Edward M. Lichten, Birmingham, MI (US)

(21) Appl. No.: 17/336,411

(22) Filed: Jun. 2, 2021

Related U.S. Application Data
(63) Continuation-in-part of application No. 16/997,408, filed on Aug. 19, 2020.
Application No. 16/997,408 is a continuation-in-part of application No. 15/456,816, filed on Mar. 13, 2017, now abandoned.
Application No. 15/456,816 is a continuation-in-part of application No. 14/035,237, filed on Sep. 24, 2013, now abandoned.
(60) Provisional application No. 61/704,797, filed on Sep. 24, 2012.
(60) Provisional application No. 61/704,797, filed on Sep. 24, 2012.

(51) Int. Cl. A61K 31/568; A61K 31/58; A61K 9/00; G01N 33/74

(52) U.S. Cl. CPC A61K 31/568; A61K 31/58; G01N 33/743; A61K 9/0019; A61K 9/0053

(57) ABSTRACT

By initially lowering the SHBG with a synthetic androgen and, thereafter, raising the testosterone level in a person's body, an effective method for treating a corona virus is achieved by first using a synthetic anabolic steroid and, thereafter, a natural androgen.

### CROSS-REFERENCE TO RELATED APPLICATIONS

This application is a continuation-in-part application of U.S. Non-Provisional patent application Ser. No. 16/997,408, filed Aug. 19, 2020 for “Use of Androgen for Treating Gastrointestinal Disorders” which, in turn, claims the priority benefit of U.S. Non-Provisional patent application Ser. No. 14/035,237, filed Sep. 24, 2013, for “Use of Androgen For Disease Treatment,” which claims the priority benefit of U.S. Provisional Patent Application Ser. No. 61/704,797, filed Sep. 24, 2012, which claims the priority benefit of U.S. patent application Ser. No. 15/456,816, filed Mar. 13, 2017, the entire disclosures of which are hereby incorporated by reference in their entirety.

### BACKGROUND OF THE INVENTION

### 1. Field of the Invention

The present invention pertains to a method for treating the corona virus COVID 19. More particularly, the present invention concerns the use of androgens to treat the COVID 19 virus. Even more particularly, the present invention pertains to the use of both synthetic and natural androgens to treat the COVID 19 virus.

### 2. Description of Related Art

In the above-referred to co-pending application there is discussed the use of an androgen, such as stanozolol, for blocking liver production of SHBG. Further, there is disclosed the fact that prior art is replete with reports showing the use of the laboratory measurement of SHBG using an androgen as a marker, sensitivity test, androgen receptor, mutation detection, and so forth. Furthermore, U.S. Pat. Nos. 7,186,706, 6,583,129, and 6,139,873 illustrate the utilization of stanozolol and other androgens in connection with SHBG testing.

The co-pending application further provides an extensive discussion of GI tract infections and diseases.

As is disclosed hereinafter, the predicate for the present invention resides in the postulate that the proper balance of natural and synthetic anabolic steroids can treat COVID 19 virus.

It is to this to which the present invention is directed.

### SUMMARY OF THE INVENTION

In accordance with the present invention, there is provided a method for treating COVID 19.

The present method comprises the steps of lowering the SHBG in the body through the utilization of a synthetic androgen and, thereafter, raising the serum testosterone level in the body through the use of a natural androgen to raise the FAI to an acceptable level.

More specifically, by utilizing selected amounts of synthetic androgens and the second natural androgens, it is possible to first lower the amount of SHBG in the body with the synthetic androgen and, once the reduced level is achieved, raising the testosterone level in the body with the natural androgen to achieve a specified FAI and an equilibrium between the SHBG and the natural androgen.

Preferably, the androgens used herein are steroids. Both naturally occurring and synthetic steroids may be used.

For a more complete understanding of the present invention, reference is made to the following detailed description of the invention.

### DETAILED DESCRIPTION OF THE INVENTION

As noted above, the present invention provides a method for treating the COVID 19 virus.

As is known to one of ordinary skill in the art, the FAI in a person is determined by a ratio of the serum testosterone level with respect to the SHBG in that person. In other words, FAI=testosterone level/SHBG.

Although not wishing to be bound by any theory, in a May 12, 2020 article from Hamburg, Germany it was reported that 90% of the men who died with COVID 19 had low testosterone. Therefore, it is hypothesized that these men had significantly reduced Estrogen Receptor-Beta and ER-Beta/ER-Alpha ratio as well.

Based on the discovery that Estrogen Receptor-Beta controls the cytokine release, and that ER-Beta is decreased in IBD, we hypothesized and proved that the proper balance of natural and synthetic anabolic steroids can bring remission by elevating the ER-Beta and ER-Beta/ER-Alpha ratio toward normal.

Thus, it is hypothesized that the same process is applicable to treating COVID 19.

Therefore, it is believed that by stabilizing the ER-Beta by first lowering cytokine levels and then mitigating the cytokine storm that is the principal cause of death the virus can be treated. The earlier the intervention with the listed anabolic steroids, and the higher the FAI, this will predict whether the COVID 19 immunological component can be reset toward normal homeostasis and improve recovery rate.

Thus, in accordance herewith, by utilizing selected amounts of a synthetic androgen, it is possible to lower the amount of SHBG in the body up to 8 fold.

Useful synthetic androgens include stanozolol and/or danazol which are used to lower the SHBG by 80% and lowers the testosterone or FAI.

Generally, the synthetic androgen is administered over a period of time of about four to about eight weeks in order to lower the SHBG to within a young adult range of about 30 isomole per liter (nmol/l) to about 45 nmol/l for a female and to within a range for an older male of about 5 nmol/l to about 15 nmol/l.

After the treatment with the synthetic androgen, which lowers the total testosterone concentration to a specified range, it is necessary to, then, add the natural androgen into the system to raise the serum testosterone to a normal level and to raise the FAI in the individual to an acceptable level.

In practicing the present invention, both natural and synthetic androgens may be used.

Representative natural androgens, used herein are, for example, dihydroepiandrosterone, androstenedione, androstenediol, androsterone, dihydrotestosterone, testosterone, nandrolone, and the like.

Synthetic androgens used herein include, in addition to the stanozolol and danazol, are for example, oxandrin, furzabolin, dianabol, anadrol, and the like.

As noted, in the practice hereof, the synthetic androgen which is a synthetic anabolic steroid is used to lower the SHBG concentration. The synthetic anabolic steroids include, for example: stanozolol, dianabol, danazol, and anadrol.

Preferably, stanozolol is used as the synthetic androgen for lowering the SHBG. The stanozolol can be administered either orally or as an injectable.

The gender of the patient or person determines the amount of natural anabolic steroid necessary to raise the testosterone level in order to achieve the desired FAI. For a male, the FAI should range from about 1 to about 2. For a female, the FAI should range from about 0.03 to about 0.10. Thus, a male needs to have an SHBG level of from about 5 nmol/l to about 15 nmol/l and a female from about 30 nmol/l to about 45 nmol/l in order to ultimately achieve the desired FAI.

Regardless of gender, generally, from about 4.0 milligrams (mgs) to about 8.0 mgs of the stanozolol is administered orally, daily, for about four to about eight weeks in order to effectuate specific lowering of the SHBG.

Where injections of the natural androgen are used, generally, from about 10.0 mgs to about 50.0 mgs are injected, once a week, for about four to about eight weeks.

As noted above, after the SHBG has been lowered with the synthetic androgen to an acceptable level, the natural androgen, such as an anabolic steroid, is, then, administered into the body to rebalance the equilibrium in the body to raise the serum testosterone and the FAI.

[symbol]

Where the patient is a male, generally from about 100 mgs to about 250 mgs of “added back” natural androgen, e.g., testosterone is administered as an injection, once a week, for about four to about eight weeks, at which time the dosage is modified to about 5 to 10 mgs to reduce side effects and optimize clinical presentation.

With a female patient, the amount of natural anabolic testosterone administered will generally range from about 15 mgs to about 70 mgs, once a week, for about four to about eight weeks.

According to the present method where the testosterone and nandralone are used in combination, generally, they will be administered in a respective weight ratio of from about 4.0:1.0 to about 1.0:1.0 of testosterone to nandralone.

The administration of the first and second androgens may be continued indefinitely as the desired levels of FAI is achieved, Preferably, periodic measurements of SHBG and testosterone levels in the patient are taken in order to determine if the desired ranges have been achieved. It is to be understood that it is well known to those of ordinary skill in the art to measure SHBG and testosterone levels in a patient using any standard blood or saliva test.

By following the steps described herein, generally, in about four to eight weeks, equilibrium between the SHBG and the testosterone is achieved, and the FAI will fall within the desired range, i.e., FAI is greater than 1. It is to be appreciated that by administering the specified amounts of androgen, described above, there is a concomitant “re-balancing” of an appropriate testosterone level in relation to the decrease in SHBG.

Although not wishing to be bound by any theory, it appears that the first or the initial insult on the immune system is the environmental toxin/xenoestrogen presence. It is a stressor, just like a viral infection that triggers an adrenal response. The body's General Adaptation Syndrome causes an outpouring of corticosteroids from the Hypothalamic-Pituitary-Adrenal Axis (HPA). This suppresses the Hypothalamic-Pituitary-Gonadal Axis (HPG) that produces testosterone.

Concurrently, estrogens and specifically xenoestrogens increase the liver production of Sex Hormone Binding Globulin (SHBG). As reported by Anderson in 1972 SHBG binds 98% of testosterone. Therefore, there is both a decrease in testosterone and an increase in SHBG. The Free Androgen Index [TT/SHBG] is the best simple readily available measurement/Laboratory Test of what is hormonally occurring.

The use of the natural androgen is directed to the loss of bioavailable testosterone [TT/SHBG]. This reduces testosterone binding to the outside of a cell (Androgen Receptor). Therefore, with testosterone binding to the cell, the testosterone present in cytoplasma is reduced and its ability to bind to the estrogen receptors on the nuclear membrane is reduced.

The addition of stanozolol decreases SHBG by about 80%, thereby increasing the FAI. Nandrolone is only 5% bonded to SHBG but has a greater affinity for SHBG by about three times more than that of testosterone. Nandrolone frees testosterone from SHBG. Thus, the combination of natural and synthetic androgens raises the FAI bioavailable testosterone which, in turn, stabilizes the Estrogen Receptor-Beta.

It is contemplated that by employing the above method to reduce high levels of SHBG in a person's body and, thereafter, raise the serum testosterone level, a satisfactory FAI can be achieved, thereby treating the COVID 19 virus, since FAI represents available testosterone in the cascade from disease to autoimmune response. As a result thereof, symptoms and side effects of gastrointestinal disorders may be managed and minimized.

Stanozolol reduces the serum concentration of SHBG by 80%. Danazol does this as well. Stanozolol is 100 times more potent mg for mg than danazol. Using stanozolol reduces side effects and hirsutism caused by danazol.

The FAI in men is typically increased 4 fold; in women it can increase by 20 fold.

It is to be understood that the androgens can be administered substantially simultaneously or sequentially of synthetic androgen followed by the natural androgen.

It is to be understood that the present invention is not for measuring the serum TT/SHBG biomarkers as the FAI, but for a method of treatment directly attributed to changes in the intracellular levels/ratio of the Estrogen Receptor-Beta that cannot be easily measured and is both time consuming and extremely expensive.

The Estrogen Receptor-Beta to Alpha Receptor ratio is a biomarker for indicating a viral disease. Therefore, it is believed that the corollary is that when the immune system is not in hemostasis that the FAI is suppressed. The FAI drives the immune system to hemostasis.

Having thus described the invention, what is claimed is:

1. A method for treating a corona virus, comprising the steps of:
(a) administering to a person a predetermined amount of a synthetic androgen over a period of time of from about four to about eight weeks to lower the SHBG level of the person to a pre-selected level;
(b) thereafter, administering a naturally occurring androgen to the person over a period of time of from about four to about eight weeks to raise the testosterone to a normal level of the person to achieve an acceptable Free Androgen Index (FAI) according to the gender of the person; and
wherein achieving an acceptable FAI minimizes the symptoms of the virus.

2. The method of claim 1 wherein the virus is COVID 19.

3. The method of claim 2 wherein the synthetic and naturally occurring androgens are steroids.

4. The method of claim 3 wherein the naturally occurring androgen is selected from the group consisting of dihydroepiandrosterone, androstenedione, androstenediol, androsterone, dihydrotestosterone, testosterone, nandrolone, oxandrin, furazabol, dioxadol, and anadrol.

5. The method of claim 3 wherein the naturally occurring androgen is selected from the group consisting of testosterone and nandrolone.

6. The method of claim 5 wherein the naturally occurring androgen is nandrolone.

7. The method of claim 5 wherein the synthetic androgen is selected from the group consisting of danazol and stanozolol.

8. The method of claim 3 wherein the natural androgen is administered orally in an amount ranging from about 10 milligrams to about 8.0 milligrams daily for about four to eight weeks.

9. The method of claim 8 wherein the person is male, and the synthetic androgen is administered as an injection in an amount ranging from about 100 milligrams to about 250 milligrams, once a week, for about four to about eight weeks.

10. The method of claim 8 wherein the person is a female and the synthetic androgen is administered as an injection in an amount which ranges from about 15 milligrams to about 70 milligrams, once a week, for about four to about eight weeks.

11. The method of claim 2 wherein the SHBG is lowered to about 5 nanomoles per liter to about 15 nanomoles per liter for a male and to about 30 nanomoles per liter to about 45 nanomoles per liter for a female.

12. The method of claim 2 wherein the FAI after treatment ranges from about 1 to about 2 for a male and from about 0.03 to about 0.10 for a female.

13. The method of claim 1 which further comprises:
determining the estrogen receptor beta to alpha ratio as a biomarker for indicating an inflammation within the gastrointestinal tract prior to steps (a) and (b).
